(19) United States
(12) Patent Application Publication
(10) Pub. No.: US 2012/0196290 A1
(43) Pub. Date: Aug. 2, 2012

(54) METHOD FOR DIAGNOSING SPINAL MUSCULAR ATROPHY

(75) Inventors: SHOU-MEI WU, Kaohsiung City (TW); CHUN-CHI WANG, Kaohsiung City (TW); JAN-GOWTH CHANG, Kaohsiung City (TW); YUH-JYH JONG, Kaohsiung City (TW)

(73) Assignee: KAOHSIUNG MEDICAL UNIVERSITY, Kaohsiung City (TW)

(21) Appl. No.: 13/015,450

(22) Filed: Jan. 27, 2011

(51) Int. Cl. C12Q 1/68

(52) U.S. Cl. 435/6.12

(57) ABSTRACT

A method for diagnosing spinal muscular atrophy is provided. The method includes providing a biological sample of a subject containing a nucleotide of SMN gene, amplifying SMN exons 1, 2a, 2b, 3, 4, 5, 6, 7, and 8 by a universal multiplex PCR using the nucleotide as a template and the primers to obtain fragments of the SMN exons 1, 2a, 2b, 3, 4, 5, 6, 7, and 8, labeling the fragments of the SMN exons 1, 2a, 2b, 3, 4, 5, 6, 7, and 8 by a fluorescent primer to obtain fluorescence-labeled exon fragments, and analyzing the fluorescence-labeled exon fragments by a capillary electrophoresis under a optimized separation condition. If the SMN1/SMN2 ratios in exon 7 and 8 are different, it indicates that the subject is susceptible to spinal muscular atrophy. Additionally, if the peak of certain exon fragment appears crossed, it indicates an intragenic mutation in the exon.

### BACKGROUND OF THE INVENTION

1. Field of the Invention

The present invention relates to diagnosis of spinal muscular atrophy, and in particular relates to a method for diagnosing spinal muscular atrophy by capillary electrophoresis.

2. Description of the Related Art

Spinal muscular atrophy (SMA) is an autosomal recessive disease characterized by degeneration of motor neurons in the anterior horn of a spinal cord, leading to muscular paralysis and atrophy. SMA is traditionally categorized into three types, according to the age and severity. For children with SMA, SMA is categorized as: type I, severe; type II, intermediate; and type III, mild. For adults with mild symptoms of SMA, SMA is categorized as type IV. Additionally, for prenatal onset of very severe symptoms of SMA and early neonatal death due to SMA, SMA is categorized as type 0 (Eur J Paediatr Neurol 1999; 3:49-51; Lancet 1995; 346:1162; Neuromuscul Disord 1992; 2:423-428). SMA occurs in approximately 1 in 6000-10000 live births and has a carrier frequency of 1 in 50. It is the second most common autosomal recessive inherited disorder in humans and the most common genetic cause of infant mortality (Semin Neurol 1998; 18:19-26).

SMA is caused by the homozygous deletion or mutations of the survival motor neuron gene (SMN) including telomeric SMN (SMN1) and centromeric SMN (SMN2) genes. The genes possess two differences, which are substitution of single nucleotides in exon 7 (c 840 C>T) and 8 (G>A) in the cDNAs. Deletion of the SMN1 gene has been reported in approximately 94% of clinically typical SMA-affected patients, and the SMN2 copy number has been found to be related to the disease severity and life length. To diagnose SMA, most techniques quantify the nucleotide difference in exon 7 of SMN1/SMN2. However, it has been found that the SMA in approximate 6% of affected patients is caused by point mutations at other exons in which SMN1 is present. Therefore, only detection of the difference of SMN1/SMN2 genes in exon 7 could not accurately diagnose the SMA disease in a clinical environment.

Thus, to improve the diagnostic accuracy of the SMA disease in a clinical environment, a novel diagnostics method and diagnosis kit is required.

### BRIEF SUMMARY OF THE INVENTION

The invention provides a method for diagnosing spinal muscular atrophy, comprising: (a) providing a biological sample comprising a nucleotide containing SMN gene, wherein the biological sample is obtained from a subject; (b) providing primers for SMN exons 1, 2a, 2b, 3, 4, 5, 6, 7, and 8; (c) amplifying SMN exons 1, 2a, 2b, 3, 4, 5, 6, 7, and 8 by a universal multiplex PCR using the nucleotide as a template and the primers to obtain fragments of the SMN exons 1, 2a, 2b, 3, 4, 5, 6, 7, and 8; (d) labeling the fragments of the SMN exons 1, 2a, 2b, 3, 4, 5, 6, 7, and 8 by a fluorescent primer to obtain fluorescence-labeled exon fragments, and (e) analyzing the fluorescence-labeled exon fragments by a capillary electrophoresis under a separation condition comprising a separation matrix composed of a copolymer of HEC (1.5%) and HPC (2.0%), an applied voltage between −5 to −10 kV, an ionic strength determined by 1.0× to 3.0 TBE buffer concentration, and a capillary temperature between 15 to 25° C., wherein different SMN1/SMN2 ratios in exon 7 and 8 indicates that the subject is susceptible to spinal muscular atrophy.

The invention also provides a kit of for assaying a sample from a subject to detect susceptibility of spinal muscular atrophy, comprising: at least one primer pairs selected from a group consisting from SEQ ID NOs: 1-2, 3-4, 5-6, 7-8, 9-10, 11-12, 13-14, 15-16, or 17-18; and a user instruction which indicates a separation condition comprising a separation matrix composed of a copolymer of HEC (1.5%) and HPC (2.0%), an applied voltage between −5 to −10 kV, an ionic strength determined by 1.0× to 3.0×TBE buffer concentration, and a capillary temperature between 15 to 25° C.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIGS. 1A-1H show various ratios of SMA1/SMN2 in exon 7 and 8 by a capillary electrophoresis analysis;

FIG. 2 shows a result of a capillary electrophoresis analysis, wherein the SMA1/SMN2 in exon 7 and 8 are 0:2 and 1:1, respectively;

FIGS. 3A-3B show results of a capillary electrophoresis analysis and DNA sequencing of an SMA patient bearing one copy of SMN1 and a c.22_{—}23insA mutation in exon 1;

FIGS. 4A-4B show results of capillary electrophoresis analysis and DNA sequencing of a healthy subject;

FIGS. 5A-5C show results of capillary electrophoresis analysis of a sample with SMA1/SMN2=1:2 and a 22_{—}23insA in exon 1 using different separation matrices in 2.0×TBE buffer (A) 1.5% HEC+2.0% HPC+5M urea; (B) 1.5% HEC+5M urea; (C) 2.0% HPC+5M urea;

FIGS. 6A-6C show results of capillary electrophoresis analysis of a heterozygote sample with a ratio of SMN1/SMN2 equaled to 1:2 and a 22_{—}23insA in exon 1 using different capillary temperatures: (A) 15° C., (B) 20° C. and (C) 25° C.;

FIGS. 7A-7C show results of capillary electrophoresis analysis of a heterozygote sample with a ratio of SMN1/SMN2 equaled to 2:1 using different applied voltages: (A) −4 kV; (B) −6 kV; (C) −8 kV and (D) −10 kV.

FIG. 8 shows results of capillary electrophoresis analysis of a normal control sample (SMN1/SMN2=2:2) after full-scale genotyping of SMN gene; and the labels above the peak indicated the exon number;

FIGS. 9A-9H show results of capillary electrophoresis analysis of individuals with different SMN1/SMN2 gene ratios from 4:0 to 0:4 using the optimized conditions.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

The invention provides a method for determining a mutation in SMN gene. The method comprises: (a) providing a biological sample comprising a nucleotide containing an SMN gene; (b) providing primers for SMN exons 1, 2a, 2b, 3, 4, 5, 6, 7, and 8; (c) amplifying SMN exons 1, 2a, 2b, 3, 4, 5, 6, 7, and 8 by a universal multiplex PCR using the nucleotide as a template and the primers to obtain fragments of the SMN exons 1, 2a, 2b, 3, 4, 5, 6, 7, and 8; (d) labeling the fragments of the SMN exons 1, 2a, 2b, 3, 4, 5, 6, 7, and 8 by a fluorescent primer to obtain fluorescence-labeled exon fragments, and (e) analyzing the fluorescence-labeled exon fragments by a capillary electrophoresis under an optimized separation condition. If ratios of SMN1/SMN2 in exon 7 and 8 are different, the result indicates that at least one gene conversion has occurred in the SMN gene. Additionally, if a crossed peak is observed in a capillary electrophoresis analysis, it indicates that at least one mutation has occurred in the SMN gene.

Firstly, a nucleotide containing SMN gene is provided. The nucleotide contains SMN exons 1, 2a, 2b, 3, 4, 5, 6, 7, and/or 8. Preferably, the nucleotide is a DNA fragment.

Primers for exons 1, 2a, 2b, 3, 4, 5, 6, 7, and 8 are provided. The primers of the invention include any kind of primer that is capable of amplifying exons 1, 2a, 2b, 3, 4, 5, 6, 7, and 8 by a polymerase chain reaction (PCR). The primers can be used in same or different PCR procedures, preferably, in same PCR procedures. In one embodiment, the primers for the SMN exons 1, 2a, 2b, 3, 4, 5, 6, 7, and 8 are SEQ ID NOs: 1-18, respectively, wherein the primers for the SMN exon 1 are SEQ ID NO: 1 and 2, the primers for the SMN exon 2a are SEQ ID NOs: 3 and 4, the primers for the SMN exon 2b are SEQ ID NOs: 5 and 6, the primers for the SMN exon 3 are SEQ ID NOs: 7 and 8, the primers for the SMN exon 4 are SEQ ID NOs: 9 and 10, the primers for the SMN exon 5 are SEQ ID NO: 11 and 12, the primers for the SMN exon 6 are SEQ ID NO: 13 and 14, the primers for the SMN exon 7 are SEQ ID NO: 15 and 16, and the primers for the SMN exon 8 are SEQ ID NO: 17 and 18.

The SMN exons 1, 2a, 2b, 3, 4, 5, 6, 7, and 8 are amplified by a universal multiplex PCR using the nucleotide as a template and the primers to obtain fragments of the SMN exons 1, 2a, 2b, 3, 4, 5, 6, 7, and 8. Subsequently, the fragments of the SMN exons 1, 2a, 2b, 3, 4, 5, 6, 7, and 8 are labeled by a fluorescent primer to obtain fluorescence-labeled exon fragments. The fluorescent primer can be SEQ ID NO: 21.

The universal multiplex PCR process includes two steps. In the first step, the SMN exons 1, 2a, 2b, 3, 4, 5, 6, 7, and 8 are amplified using the primers (SEQ ID NOs: 1-18) to obtain fragments of the SMN exons 1, 2a, 2b, 3, 4, 5, 6, 7, and 8. In the second step, the fragments of the SMN exons 1, 2a, 2b, 3, 4, 5, 6, 7, and 8 then are amplified and labeled using a forward fluorescent primer (5′) (SEQ ID NO: 21) and reverse primer (3′).

The amount of PCR cycles in the first step is not limited. A minimal amount of cycles may be used. The amount of cycles can be less than 10, preferably less than 2-5 to roughly amplify the SMN exons 1, 2a, 2b, 3, 4, 5, 6, 7, and 8. However, in the second step, the amount of PCR cycles preferably is more than 25 to produce enough amounts of fluorescence-labeled exon fragments. The PCR product can be directly analyzed by a capillary electrophoresis without any treatments.

The fluorescent molecules of the invention can be any suitable molecules. The fluorescent molecules includes, but is not limited to, Cy3, Cy5, FAM, HEX, TET, TAMRA, SyBr Green, GFP, or EGFP, etc. A suitable fluorescent molecule can bind to a universal primer to form the fluorescent primer, and the fluorescent primer is used to obtain the fluorescence-labeled exon fragments by PCR.

Finally, the fluorescence-labeled exon fragments are analyzed by a capillary electrophoresis. A gene which is constantly expressed can be used as an internal control for capillary electrophoresis quantitative analysis. The gene can be globin, actin, or BMP gene. To optimize the separation efficiency and resolution, the capillary electrophoresis can be performed under a specific separation condition includes, but is not limited to, separation matrix, capillary temperature, applied voltage, and ionic strength. In one embodiment, the separation matrix is composed of HEC (1.5%) polymer, HPC (2.0%) polymer, or a copolymer of HEC (1.5%) and HPC (2.0%), the applied voltage is between −5 to −10 kV, the ionic strength determined by 1.0× to 3.0×TBE buffer concentration, and the capillary temperature is between 15 to 25° C. In a preferred embodiment, the separation matrix is composed of a copolymer of HEC (1.5%) and HPC (2.0%), the applied voltage is −6 kV, the ionic strength is determined by 2.0×TBE buffer concentration, and the capillary temperature is about 15° C. If the ratios of SMN1/SMN2 in exon 7 and 8 are different, it indicates that at least one gene conversion has occurred in SMN gene. Additionally, if a crossed peak is observed in the capillary electrophoresis results, it indicates at least one mutation has occurred in SMN gene.

The invention also provides a method for diagnosing spinal muscular atrophy. The method comprises: (a) providing a biological sample comprising a nucleotide containing SMN gene, wherein the biological sample is obtained from a subject; (b) providing primers for SMN exons 1, 2a, 2b, 3, 4, 5, 6, 7, and 8; (c) amplifying SMN exons 1, 2a, 2b, 3, 4, 5, 6, 7, and 8 by a universal multiplex PCR using the nucleotide as a template and the primers to obtain fragments of the SMN exons 1, 2a, 2b, 3, 4, 5, 6, 7, and 8; (d) labeling the fragments of the SMN exons 1, 2a, 2b, 3, 4, 5, 6, 7, and 8 by a fluorescent primer to obtain fluorescence-labeled exon fragments, and (e) analyzing the fluorescence-labeled exon fragments by a capillary electrophoresis. To optimize the separation efficiency and resolution, the capillary electrophoresis can be performed under a specific separation condition includes, but is not limited to, separation matrix, capillary temperature, applied voltage, and ionic strength.

Accordingly, in one embodiment of the present invention, the separation matrix is composed of HEC (1.5%) polymer, HPC (2.0%) polymer, or a copolymer of HEC (1.5%) and HPC (2.0%), the applied voltage is between −5 to −10 kV, the ionic strength is determined by 1.0× to 3.0×TBE buffer concentration, and the capillary temperature is between 15 to 25° C. In a preferred embodiment, the separation matrix is composed of a copolymer of HEC (1.5%) and HPC (2.0%), the applied voltage is −6 kV, the ionic strength is determined by 2.0×TBE buffer concentration, and the capillary temperature is 15° C. If the ratios of SMN1/SMN2 in exon 7 and 8 are different, it indicates that the subject is susceptible to spinal muscular atrophy. Additionally, if a bifurcated peak is observed in the results of a capillary electrophoresis, it also indicates that the subject is susceptible to spinal muscular atrophy.

The “subject” of the invention refers to human or non-human mammal, e.g. a dog, a cat, a mouse, a rat, a cow, a sheep, a pig, a goat, or a primate, and expressly includes laboratory mammals, livestock, and domestic mammals. In one embodiment, the mammal may be a human; in others, the mammal may be a rodent, such as a mouse or a rat. In another embodiment, the subject is an animal model (e.g., a transgenic mouse model) of SMA. Alternatively, the subject is an SMA patient. The SMA patient can be homozygous or heterozygous for mutations in SMN1. The subject can be an adult, child, infants or fetus.

The “biological sample” of the invention can be isolated or collected from any source which contains genomic DNA, such as a blood sample, sample of amniotic fluid, sample of cerebrospinal fluid, or tissue sample from skin, muscle, buccal or conjunctival mucosa, placenta, gastrointestinal tract or other organs and tissues.

The “HEC” herein refers to hydroxyethyl cellulose and any of the forms of the same in the medium being considered. The “HPC” herein refers to hydroxypropyl cellulose and any of the forms of the same in the medium being considered.

The “ionic strength” can be determined by the concentration of dissolved ions in the buffer and will affect viscosity of the polymer. In higher TBE buffer concentration, the polymer solution become viscous and results in the difficulty of injection into capillaries.

The “applied voltage” is an electric field that applied between the source and destination of the electrophoresis process and supplied to the electrodes by the high-voltage power supply. The applied voltage can affect speed of DNA sample movement and resolution. Higher applied voltage made DNA samples move rapidly; however, the resolution might be poor.

The “capillary temperature” can affect both viscosity of the polymer and DNA conformation. According to examples of the present invention, one skilled in the art can easily recognizes that the mutation in SMN gene can be better resolved if an even lower temperature can be employed.

When the ratios of SMN1/SMN2 in exon 7 and 8 are different, it indicates that gene conversion has occurred in SMN gene. Additionally, the ratios of SMN1/SMN2 in exon 7 or 8 can be used to determine whether the subject is a healthy subject, SMA carrier, or SMA patient.

Generally, there are two copy of normal SMN1 in healthy subjects. For example, the SMN1/SMN2 ratio of healthy subjects may be 2:2, 2:1, 3:1, 2:0, 3:0, 3:2, 4:0, or 2:3, etc. Alternatively, there is only one copy of normal SMN1 in SMA carriers. For example, the SMN1/SMN2 ratio of SMA carriers may be 1:1, 1:2, 1:3 or 1:4, etc. However, there is no normal SMN1 in SMA patients. For example, the SMN1/SMN2 ratio of SMA patients may be 0:2, 0:3, or 0:4, etc. Thus, a subject can be determined by the ratio of SMN1/SMN2 to determine whether the subject is a healthy subject, SMA carrier, or SMA patient.

The SMN1/SMN2 ratios described above normally occur in most subjects. However, in a few subjects, mutations occur in SMN exon resulting in the loss of SMN gene function. For example, in one SMA patient, its SMN1/SMN2 ratio in SMN exon 7 and 8 are 1:2 and 1:3, respectively, and at least one mutation is observed in other SMN exons. This indicates that the SMA patient is due to the mutation of other SMN exons, but not SMN/deletion.

In the invention, the capillary electrophoresis not only simultaneously analyzes the SMN1/SMN2 ratio in exon 7 and 8, but also determines the sequence difference and mutation location in other SMN exons. In addition, the PCR product can be directly analyzed by capillary electrophoresis without any treatment.

The invention further provides a kit for diagnosing susceptibility of spinal muscular atrophy. The kit comprises at least one primer pair selected from a group consisting from SEQ ID NOs: 1-2, 3-4, 5-6, 7-8, 9-10, 11-12, 13-14, 15-16, or 17-18, and a user instruction indicates the optimized separation condition, wherein the separation matrix is composed of HEC (1.5%) polymer, HPC (2.0%) polymer, or a copolymer of HEC (1.5%) and HPC (2.0%), the applied voltage is between −5 to −10 kV, the ionic strength is determined by 1.0× to 3.0×TBE buffer concentration, and the capillary temperature is between 15 to 25° C. In a preferred embodiment, the separation matrix is composed of a copolymer of HEC (1.5%) and HPC (2.0%), the applied voltage is −6 kV, the ionic strength is determined by 2.0×TBE buffer concentration, and the capillary temperature is 15° C.

Moreover, the kit of the invention further includes an internal control primer pair which is SEQ ID NOs: 19-20 or 19-22, and a fluorescent primer which is SEQ ID NO: 21.

### EXAMPLES

### Example 1

### Diagnosis of Spinal Muscular Atrophy

Eleven primer pairs were provided in Example 1 to simultaneously amplify nucleotide fragments of SMN exons 1, 2a, 2b, 3, 4, 5, 6, 7, 8, and globin gene. The amount of nucleotide fragments of the globin gene was used as an internal control. The primer pairs are shown in Table 1.

TABLE 1
length and sequence in various primer pairs

| | | Length | | Length of |
|---|---|---|---|---|
| | | of | | DNA |
| Genes | Primers | primers | SEQ ID NO | fragment |
| SMN-exon 1 | Uni-5′-exon 1 | 41 | SEQ ID NO: 1 | 308 |
| | 3′-exon 1 | 20 | SEQ ID NO: 2 | |
| SMN-exon 2a | Uni-5′-exon 2a | 44 | SEQ ID NO: 3 | 274 |
| | 3′-exon 2a | 23 | SEQ ID NO: 4 | |
| SMN-exon 2b | Uni-5′-exon 2b | 44 | SEQ ID NO: 5 | 331 |
| | 3′-exon 2b | 21 | SEQ ID NO: 6 | |
| SMN-exon 3 | Uni-5′-exon 3 | 42 | SEQ ID NO: 7 | 382 |
| | 3′-exon 3 | 23 | SEQ ID NO: 8 | |
| SMN-exon 4 | Uni-5′-exon 4 | 42 | SEQ ID NO: 9 | 439 |
| | 3′-exon 4 | 21 | SEQ ID NO: 10 | |
| SMN-exon 5 | Uni-5′-exon 5 | 42 | SEQ ID NO: 11 | 474 |
| | 3′-exon 5 | 20 | SEQ ID NO: 12 | |
| SMN-exon 6 | Uni-5′-exon 6 | 43 | SEQ ID NO: 13 | 510 |
| | 3′-exon 6 | 23 | SEQ ID NO: 14 | |
| SMN-exon 7 | Uni-5′-exon 7 | 42 | SEQ ID NO: 15 | 577 |
| | 3′-exon 7 | 21 | SEQ ID NO: 16 | |
| SMN-exon 8 | Uni-5′-exon 8 | 46 | SEQ ID NO: 17 | 809 |
| | 3′-exon 8 | 20 | SEQ ID NO: 18 | |
| β-globin (IS1) | Uni-5′-globin | 41 | SEQ ID NO: 19 | 715 |
| | 3′-globin 1 | 28 | SEQ ID NO: 20 | |
| B-globin (IS2) | Uni-5′-globin | 41 | SEQ ID NO: 19 | 763 |
| | 3′-globin 2 | 23 | SEQ ID NO: 22 | |
| fluorescent | 5′-FAM | 17 | SEQ ID NO: 21 | |
| primer | | | | |

All primer pairs were classified into two (A and B) groups. The primer pairs had the same PCR procedures in the same group. The PCR procedures of two groups are shown in Table 2.

TABLE 2
Reactant concentration in PCR reaction

| Group A | | | Group B | | |
|---|---|---|---|---|---|
| DNA | 100 | ng | DNA | 100 | ng |
| dNTP | 200 | μg | dNTP | 200 | μM |
| 10 × PCR buffer | 2.5 | μl | 10 × PCR buffer | 2.5 | μl |
| Uni-5′-exon 1 | 0.004 | μM | Uni-5′-exon 3 | 0.012 | μM |
| Uni-5′-exon 2a | 0.006 | μM | Uni-5′-exon 4 | 0.012 | μM |
| Uni-5′-exon 2b | 0.012 | μM | Uni-5′-exon 6 | 0.008 | μM |
| Uni-5′-exon 5 | 0.012 | μM | Uni-5′-exon 8 | 0.02 | μM |
| Uni-5′-exon 7 | 0.02 | μM | Uni-5′-globin | 0.006 | μM |
| Uni-5′-globin | 0.012 | μM | 3′-exon 3 | 0.08 | μM |
| 3′-exon 1 | 0.08 | μM | 3′-exon 4 | 0.16 | μM |
| 3′-exon 2a | 0.06 | μM | 3′-exon 6 | 0.08 | μM |
| 3′-exon 2b | 0.16 | μM | 3′-exon 8 | 0.16 | μM |
| 3′-exon 5 | 0.12 | μM | 3′-globin-2 | 0.06 | μM |
| 3′-exon 7 | 0.28 | μM | 5′-FAM | 0.4 | μM |
| 3′-globin-1 | 0.16 | μM | Taq | 0.5 | unit |
| 5′-FAM | 0.4 | μM | | | |
| Taq | 0.5 | unit | | | |
| Total volume | 25 | μl | Total volume | 25 | μl |

The PCR reaction was divided into two steps. In the first step, the PCR procedures were 1 cycle of 95° C. for 10 min, and 3 cycles of 95° C. for 45 sec and 60° C. for 2 min. The forward and reverse primers (as shown in Table 1) were used to amplify the SMN gene. Since the primer contains a conserved sequence, all amplified SMN gene fragments had the conserved sequence. In the second step, the universal-5′-FAM primer and a reverse primer were used to amplify and label the SMN gene fragments. After PCR reaction, the fluorescence-labeled SMN gene fragments were directly analyzed by capillary electrophoresis without any treatment. The PCR procedures of the second step were 25 cycles of 95° C. for 45 sec, 50° C. for 1.5 min and 72° C. for 1 min; and 1 cycle of 72° C. for 10 min.

In the capillary electrophoresis, Beckman P/ACE MDQ system, a laser induced fluorescence (LIF), and a DB-17 capillary with 100 μm id and 30 cm effective length were used. The LIF has an excitation wavelength of 488 nm and emission wavelength of 520 nm.

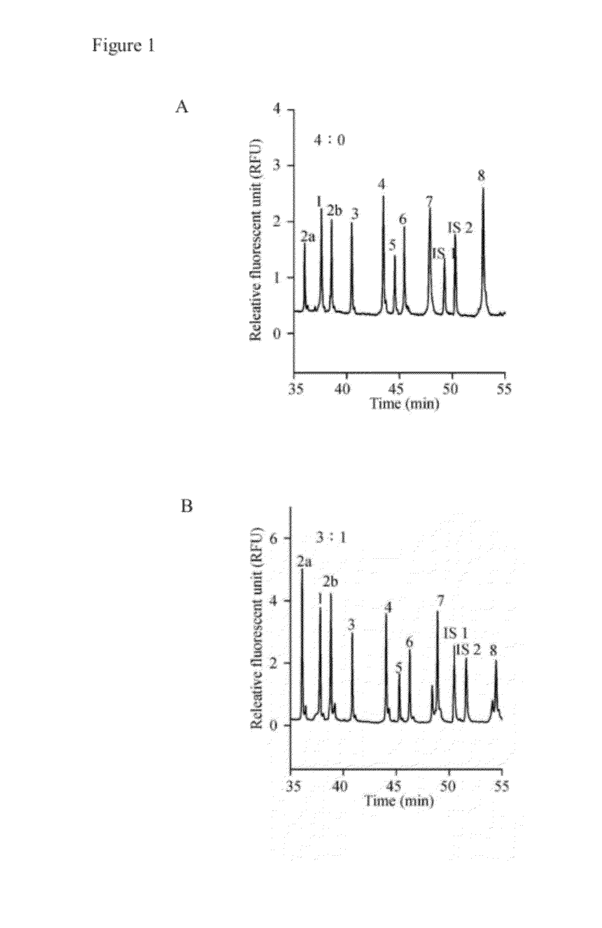

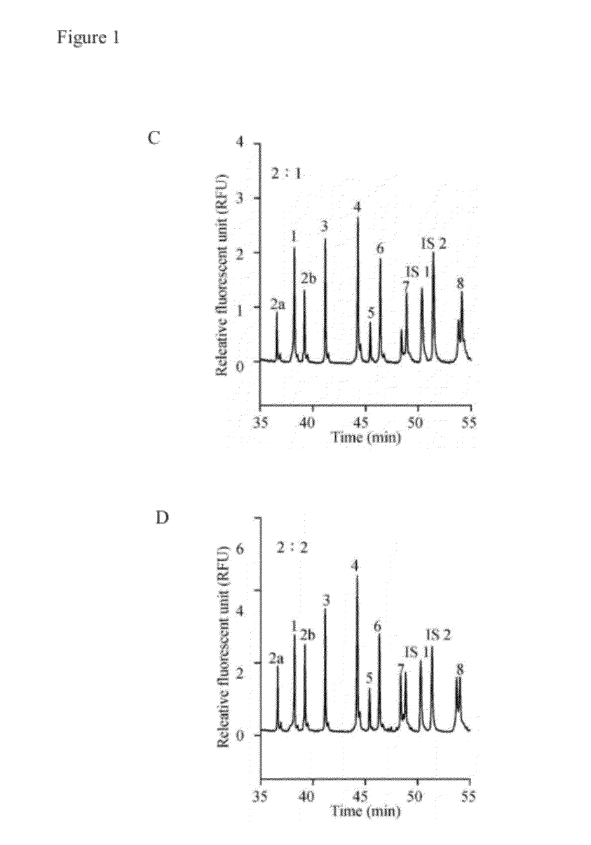

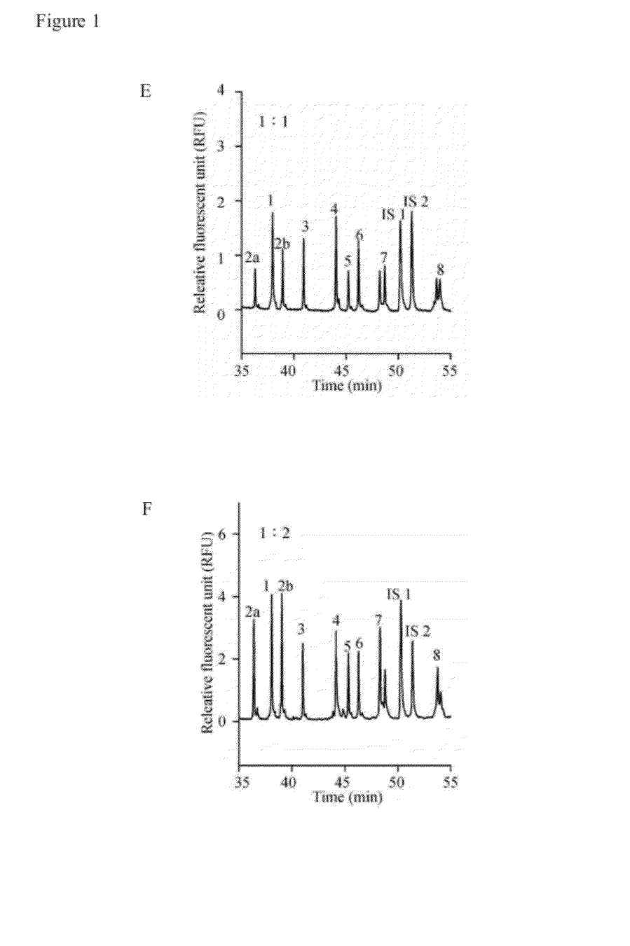

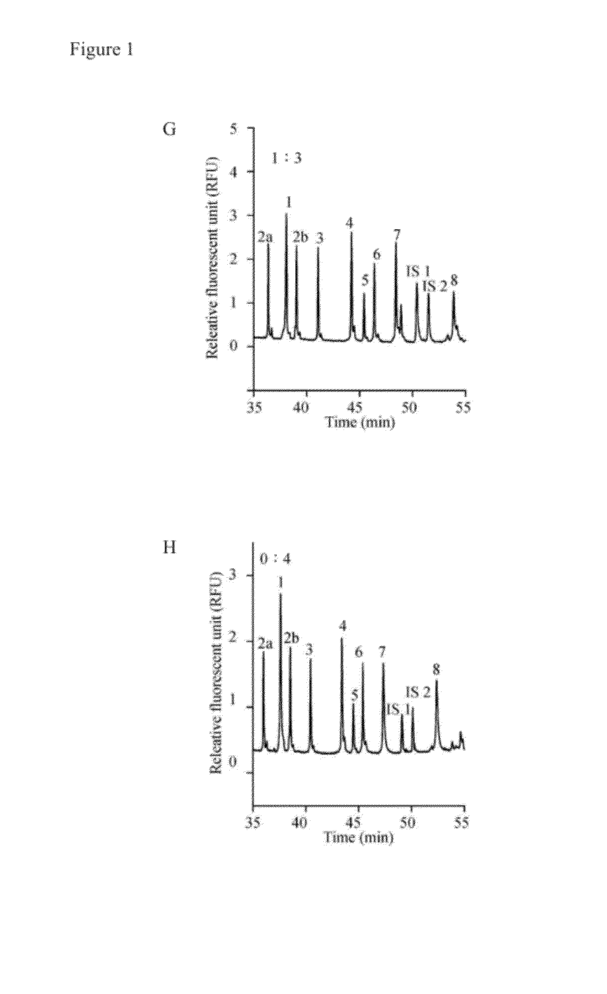

After capillary electrophoresis analysis, the nucleotide fragments of SMN exons 1, 2a, 2b, 3, 4, 5, 6, 7, and 8 were quantified. The β-globin (IS1) was used to quantify the SMN1/SMN2 ratio in exon 7, and the β-globin (IS2) was used to quantify the SMN1/SMN2 ratio in exon 8. FIGS. 1a-1h show the SMN1/SMN2 ratios of 4:0 (FIG. 1A), 3:1 (FIG. 1B), 2:1 (FIG. 1C), 2:2 (FIG. 1D), 1:1 (FIG. 1E), 1:2 (FIG. 1F), 1:3 (FIG. 1G), and 0:4 (FIG. 1H).

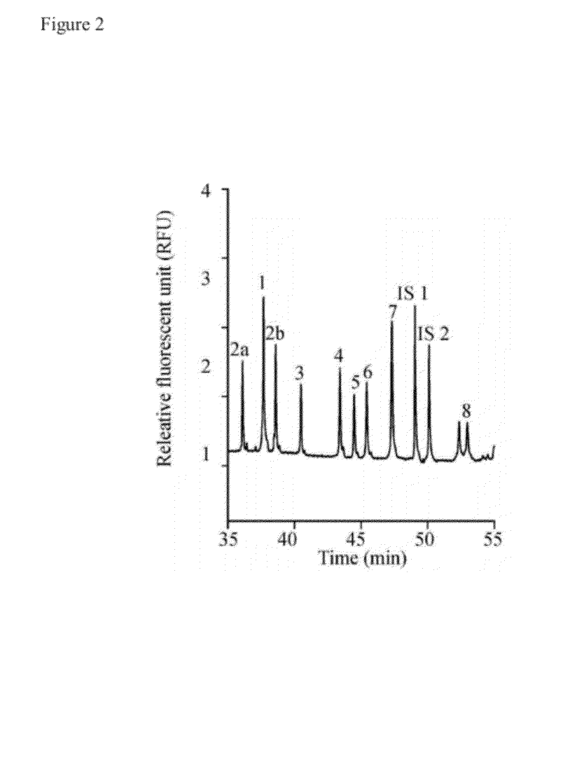

When SMN1/SMN2 ratios in exon 7 and exon 8 are different, it indicates that the gene conversion has occurred in SMN gene. Referring to FIG. 2, the SMN1/SMN2 ratio in exon 7 was 0:2 and the SMN1/SMN2 ratio in exon 8 was 1:1. The results indicated the subject was an SMA patient, and one copy of SMN2 in exon 8 was conversed to SMN 1.

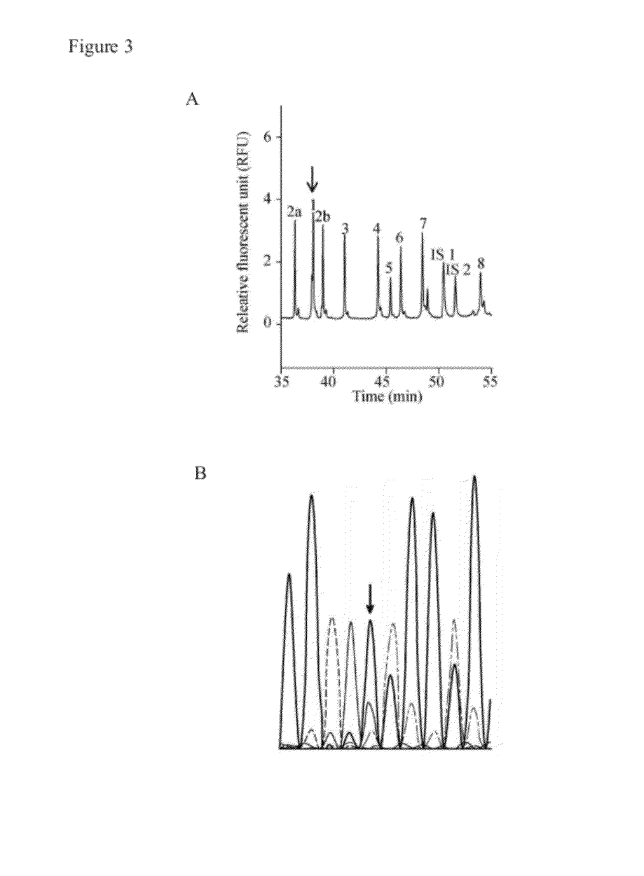

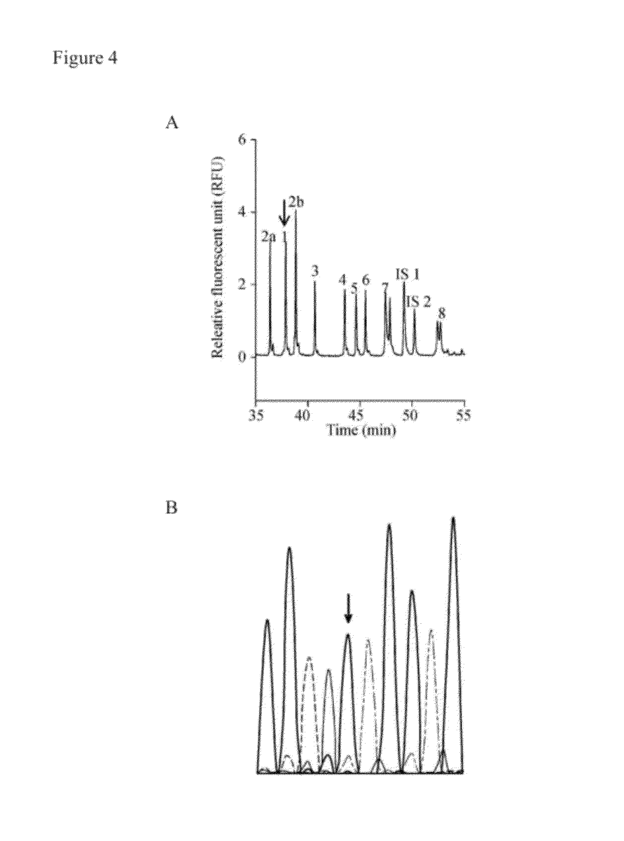

In addition to the quantitative analysis of SMN1/SMN2 in exons 7 and 8, the point mutation or difference in other SMN exons was also determined. FIGS. 3A-3B show results of capillary electrophoresis analysis and DNA sequencing of a SMA patient bearing one copy of SMN1 and a c.22_{—}23insA mutation in exon1. FIG. 4A-4B show capillary electrophoresis results and DNA sequencing of a healthy subject. When comparing FIGS. 3A and 4A, a crossed peak was observed in exon 1, and the location of mutation was determined by the sequencing data of FIGS. 3B and 4B. Referring to FIGS. 3A-3B, SMN1/SMN2 ratios in exon 7 and exon 8 were 1:3. However, an intragenic mutation was observed in exon 1, and the location of the intragenic mutation was located by sequencing data. Accordingly, the results indicated that the SMA patient had one copy of SMN1 due to a gene mutation in an SMN exon 1.

### Example 2

### Separation Conditions Optimization

To optimize the separation efficiency, the most difficult to resolve sample (DNA sample with a c.22_{—}23insA in exon 1) was used to evaluate the separation conditions. Several parameters were investigated, including separation matrix, capillary temperature, applied voltage and ionic strength.

A copolymer of HEC (1.5%) and HPC (2.0%) was initially employed for the analysis of the nine exons of the SMN gene (FIG. 5A). The separation polymer matrix could be used for recognition of several intragenic mutations. The polymer mixture was compared with the single polymers, 1.5% HEC or 2.0% HPC, for separation of the same DNA sample. Poor resolutions of SMN1/SMN2 in exon 7/8 and the intragenic mutation in exon 1 were shown in FIGS. 5B and 5C. Therefore, the polymer mixture of HEC (1.5%) and HPC (2.0%) was chosen as the separation matrix.

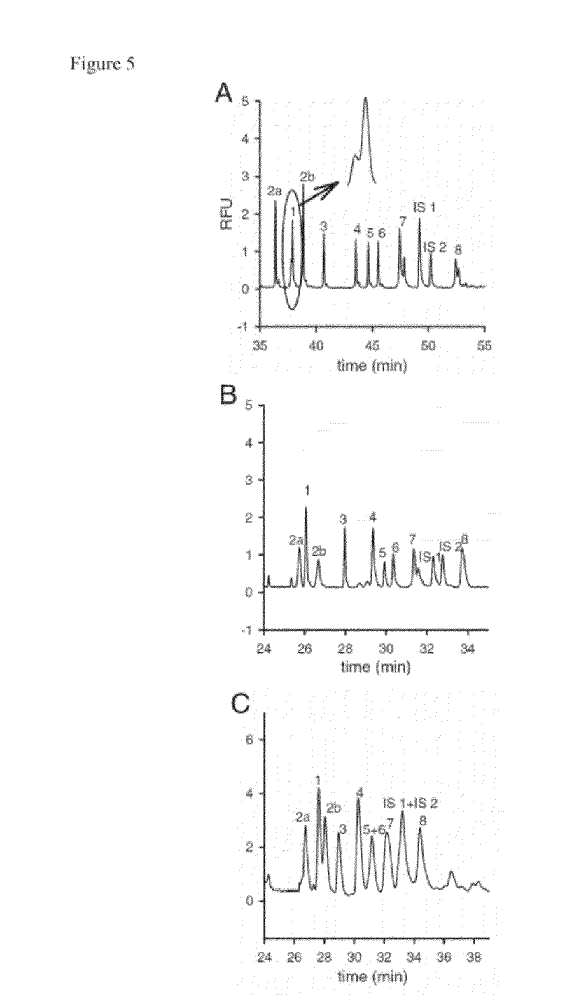

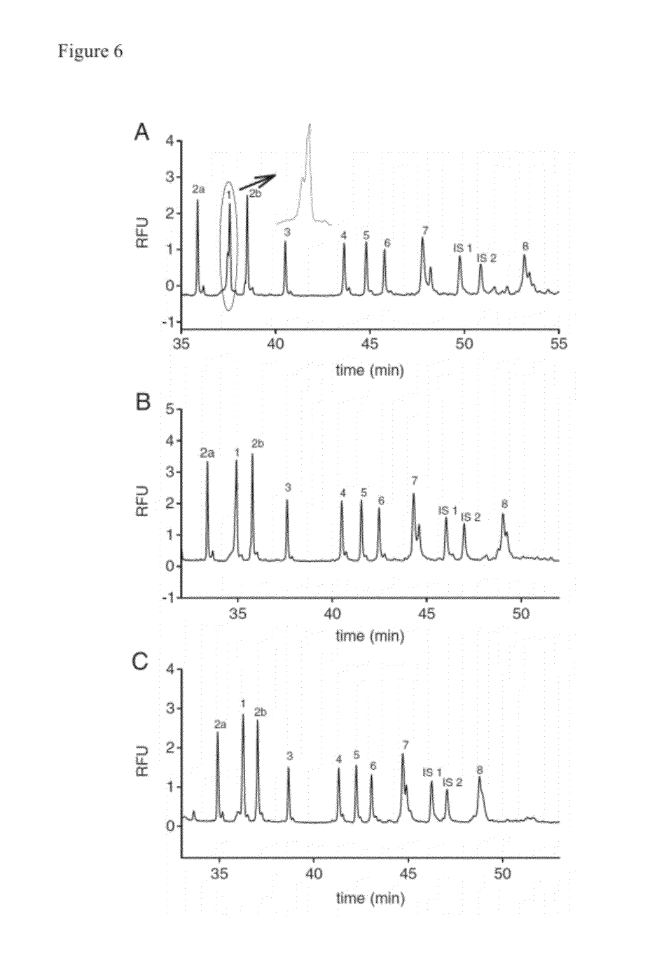

Temperature can affect both the viscosity of the polymer and the DNA conformation, thus the effect of capillary temperature on this separation method was investigated. The DNA analysis was performed at 15, 20 and 25° C., limited on the low end by the instrumentation of the Bechman P/ACE MDQ system. The resolution of the SMN1/SMN2 genes in exon 7/8 and the intragenic mutation of c.22_{—}23insA in exon 1 were optimal at 15° C. as shown in FIGS. 6A-6C. These results suggest that the mutations could be better resolved if an even lower temperature could be employed.

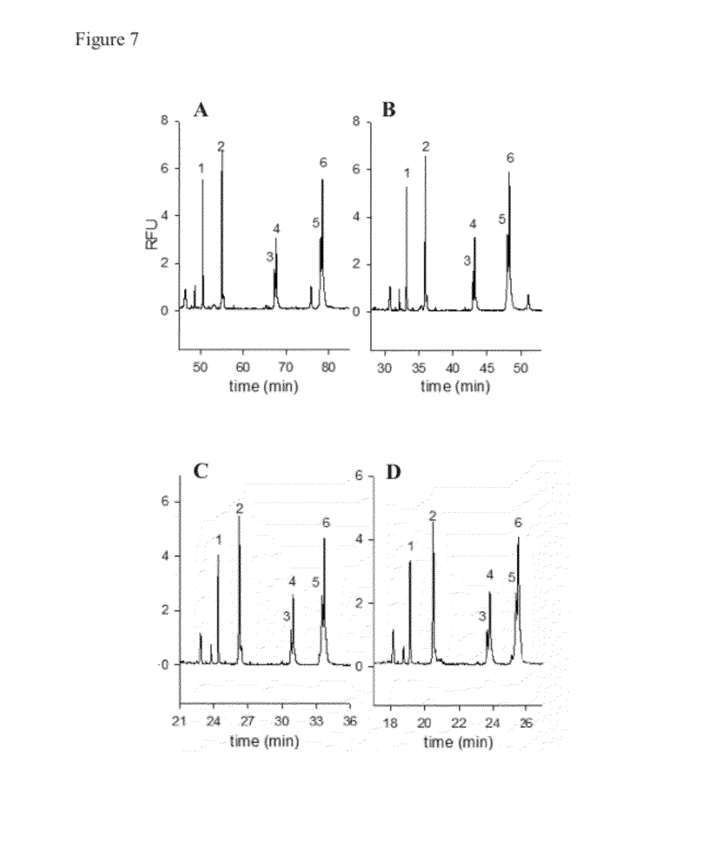

Ionic strength plays an important role in CE, thus different TBE buffer concentrations (1.0×, 1.5×, 2.0× and 2.5×) were also examined. In higher TBE concentration, the polymer solution became viscous and resulted in the difficulty of injection. The viscosity, operation and resolution of intragenic mutations had to be optimized. Finally, a 2.0×TBE solution was selected as the buffer. Higher voltage made DNA samples move rapidly; however, the resolution might be poor. Several separation voltages were investigated (−4, −6, −8 and −10 kV). The mutation in exon 1 (c.22_{—}23insA) was not well separated at −8 and −10 kV (FIG. 7A-7D). Considering the speed and resolution, −6 kV was chosen for the analysis.

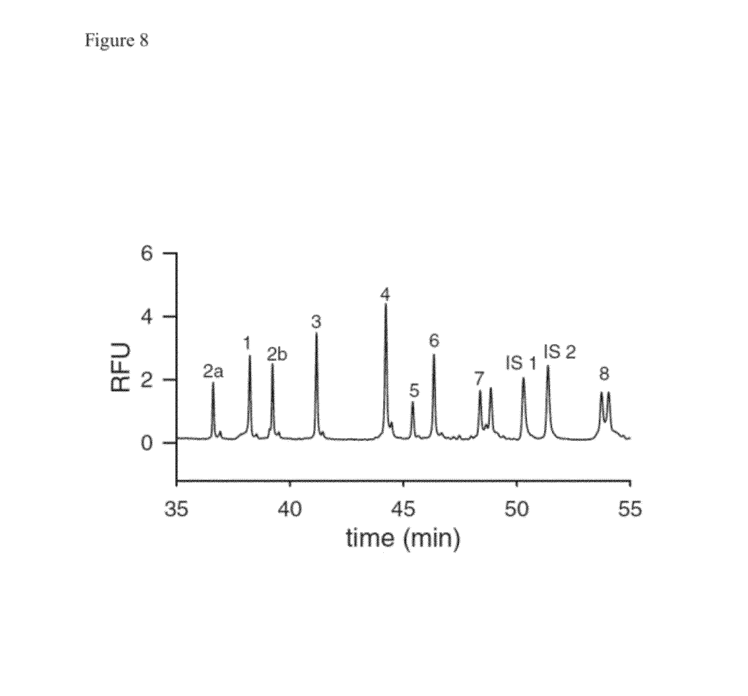

The CE system parameters were optimized, and the best separation was achieved using a polymer mixture as well as the lowest capillary temperature permitted by the instrument. In the optimized situation, all nine exons of SMN gene and two ISs were well separated, including resolution of SMN1/SMN2 in exons 7 and 8 (FIG. 8). The relative standard deviation (R.S.D.) values of migration time of eleven gene fragments were below 2.37% (n=3). This temperature was significantly lower than that in a traditional constant denaturant CE. The addition of high concentration of urea in the sample provided sufficient difference to resolve the intragenic mutations and the two SMN gene fragments in exons 7 and 8. When the temperature was decreased to 15° C., the network of the sieving matrix became more cross-connected and the viscosity increased. Those choices were beneficial for the resolution of SMN1/SMN2 and intragenic mutations. In addition, the universal fluorescent PCR was an ingenious and convenient method with only one universal fluorescence-labeled PCR primer required for all the genes when LIF was used for detection. Moreover, the PCR products could be directly analyzed by the CE method without enzyme cutting or probe hybridization when high separation efficiency was acquired.

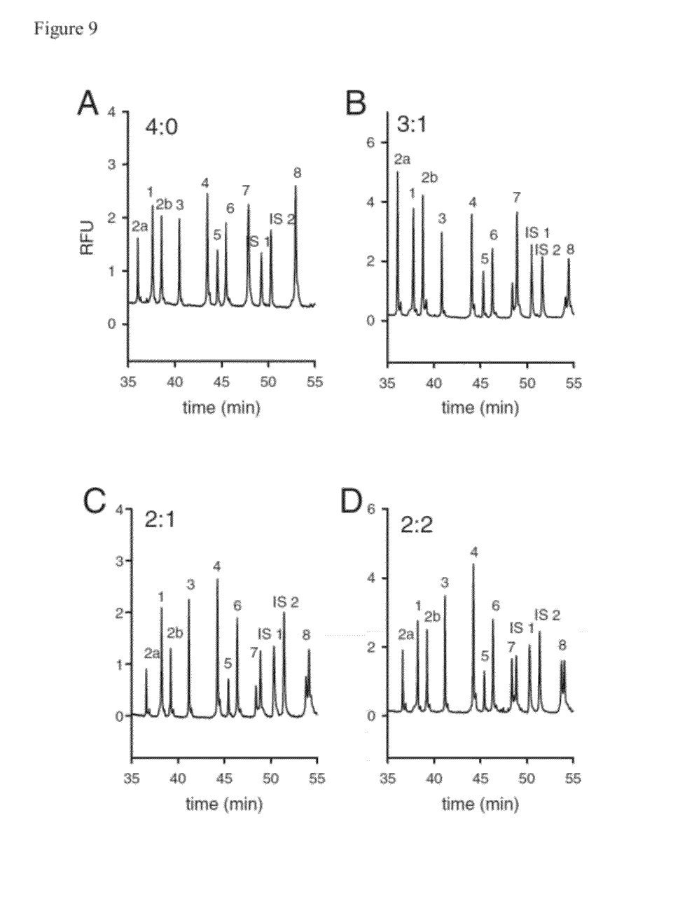

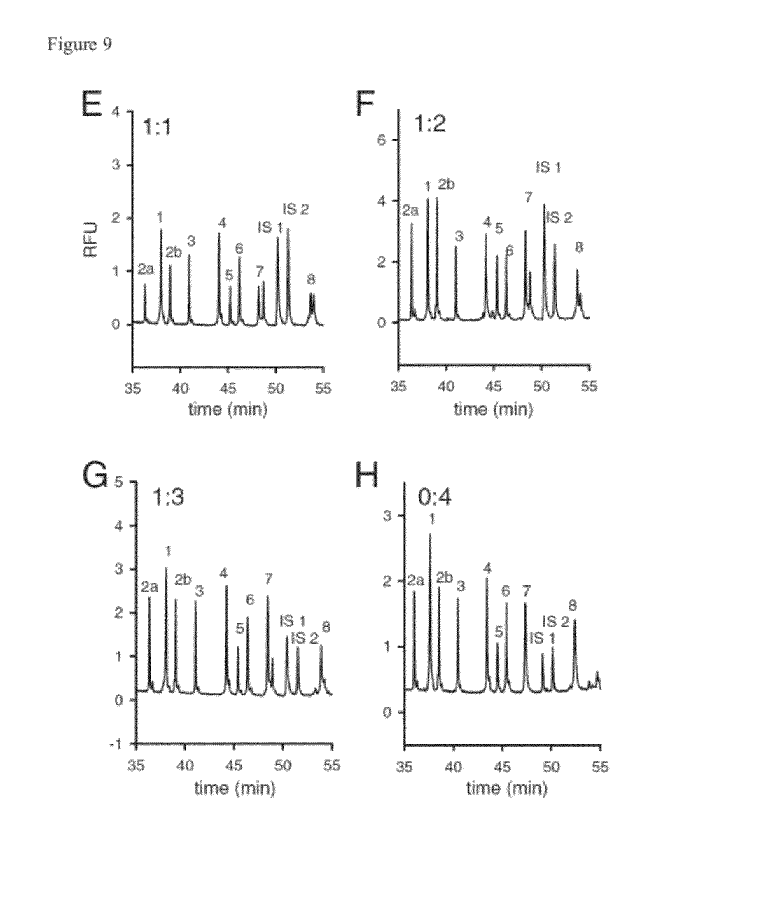

This method was further used for screening individuals, including quantification of the SMN1/SMN2 in exons 7 and 8, allowing identification of the 94% of SMA patients in which the SMN1 was deletion or gene conversion as well as detection of intragenic mutations in all nine exons allowing identification of the 6% of SMA patients having one or more copies of the SMN1 gene in which point mutation or small deletion is present. In this invention, the copy numbers of SMN1/SMN2 genes in exons 7 as well as 8 were obtained by comparison of the exon peak heights to those of the IS genetic fragments (exon 7 was compared with IS 1; exon 8 was compared with IS 2). The CE separation allowed differentiation of all ratios of SMN1/SMN2 (4:0-0:4) present in the sample population as shown in FIG. 9.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A method for diagnosing spinal muscular atrophy, comprising:
(a) providing a biological sample comprising a nucleotide containing SMN gene, wherein the biological sample is obtained from a subject
(b) providing primers for SMN exons 1, 2a, 2b, 3, 4, 5, 6, 7, and 8;
(c) amplifying SMN exons 1, 2a, 2b, 3, 4, 5, 6, 7, and 8 by a universal multiplex PCR using the nucleotide as a template and the primers to obtain fragments of the SMN exons 1, 2a, 2b, 3, 4, 5, 6, 7, and 8;
(d) labeling the fragments of the SMN exons 1, 2a, 2b, 3, 4, 5, 6, 7, and 8 by a fluorescent primer to obtain fluorescence-labeled exon fragments, and
(e) analyzing the fluorescence-labeled exon fragments by a capillary electrophoresis under a separation condition comprising a separation matrix composed of a copolymer of HEC (1.5%) and HPC (2.0%), a applied voltage between −5 to −10 kV, an ionic strength determined by 1.0× to 3.0×TBE buffer concentration, and a capillary temperature between 15 to 25° C., wherein different SMN1/SMN2 ratios in exon 7 and 8 indicates that the subject is susceptible to spinal muscular atrophy.

2. The method of claimed 1, wherein the applied voltage is −6 kv.

3. The method of claimed 1, wherein the ionic strength determined by 2.0×TBE buffer concentration.

4. The method of claimed 1, wherein the capillary temperature is 15° C.

5. The method of claim 1, wherein the different SMN1/SMN2 ratios in exon 7 and 8 indicates that at least one gene conversion occurs in SMN gene.

6. The method of claim 1, wherein the presence of a crossed peak in exons indicates that the subject is susceptible to spinal muscular atrophy.

7. The method of claim 6, wherein the presence of a crossed peak in exons indicates that at least one mutation occurs in SMN gene.

8. The method of claim 1, wherein the procedures of the universal multiplex PCR is 1 cycle of 95° C. for 10 min; 3 cycles of 95° C. for 45 sec and 60° C. for 2 min; 25 cycles of 95° C. for 45 sec, 50° C. for 1.5 min and 72° C. for 1 min; and 1 cycle of 72° C. for 10 min.

9. The method of claim 1, wherein the fluorescence-labeled exon fragments are obtained by a PCR using a fluorescent primer.

10. The method of claim 1, wherein the primers of the SMN exon 1 comprises SEQ ID NO: 1 and 2.

11. The method of claim 1, wherein the primers of the SMN exon 2a comprises SEQ ID NO: 3 and 4.

12. The method of claim 1, wherein the primers of the SMN exon 2b comprises SEQ ID NO: 5 and 6.

13. The method of claim 1, wherein the primers of the SMN exon 3 comprises SEQ ID NOs: 7 and 8.

14. The method of claim 1, wherein the primers of the SMN exon 4 comprises SEQ ID NOs: 9 and 10.

15. The method of claim 1, wherein the primers of the SMN exon 5 comprises SEQ ID NOs: 11 and 12.

16. The method of claim 1, wherein the primers of the SMN exon 6 comprises SEQ ID NOs: 13 and 14.

17. The method of claim 1, wherein the primers of the SMN exon 7 comprises SEQ ID NOs: 15 and 16.

18. The method of claim 1, wherein the primers of the SMN exon 8 comprises SEQ ID NOs: 17 and 18.

19. The method of claim 1, wherein the biological sample comprises a blood sample, an amniotic fluid, an cerebrospinal fluid, a tissue sample from skin, muscle, buccal, conjunctival mucosa, placenta, or gastrointestinal tract.

20. The method of claim 1, wherein the subject is a mammalian.

21. A kit of for assaying a sample from a subject to detect a susceptibility of spinal muscular atrophy, comprising:
at least one primer pair selected from a group consisting from SEQ ID NOs: 1-2, 3-4, 5-6, 7-8, 9-10, 11-12, 13-14, 15-16, or 17-18, and
a user instruction which indicates a separation condition comprising a separation matrix composed of a copolymer of HEC (1.5%) and HPC (2.0%), an applied voltage between −5 to −10 kV, an ionic strength determined by 1.0× to 3.0×TBE buffer concentration, and a capillary temperature between 15 to 25° C. for capillary electrophoresis.

22. The kit of claim 21, further comprising internal control primer pairs, wherein the internal control primer pair is SEQ ID NOs: 19-20 or 19-22.

23. The kit of claim 21, further comprising a fluorescent primer, wherein the fluorescent primer is SEQ ID NO: 21.


SEQUENCE LISTING (22 sequences)

SEQ ID NO: 1 - DNA, 41 bp, Artificial Sequence
  Synthetic nucleotide
ataagtgacgtactagcaacgccacaaatgtgggagggcga

SEQ ID NO: 2 - DNA, 20 bp, Artificial Sequence
  Synthetic nucleotide
gggtgctgagagcgctaata

SEQ ID NO: 3 - DNA, 44 bp, Artificial Sequence
  Synthetic nucleotide
ataagtgacgtactagcaacggtttcctgtggctttatttagga

SEQ ID NO: 4 - DNA, 23 bp, Artificial Sequence
  Synthetic nucleotide
cgagaaaataagaaaacgactaa

SEQ ID NO: 5 - DNA, 44 bp, Artificial Sequence
  Synthetic nucleotide
ataagtgacgtactagcaacgaaaagagaaaataggtgctttct

SEQ ID NO: 6 - DNA, 21 bp, Artificial Sequence
  Synthetic nucleotide
acactattaaataaggactaa

SEQ ID NO: 7 - DNA, 42 bp, Artificial Sequence
  Synthetic nucleotide
ataagtgacgtactagcaacgagcctttctcattacatcatt

SEQ ID NO: 8 - DNA, 23 bp, Artificial Sequence
  Synthetic nucleotide
tgaatttttttttttttgtatcc

SEQ ID NO: 9 - DNA, 42 bp, Artificial Sequence
  Synthetic nucleotide
ataagtgacgtactagcaacgccttataacaaaaacctgcat

SEQ ID NO: 10 - DNA, 21 bp, Artificial Sequence
  Synthetic nucleotide
aaaagttaactggataaatct

SEQ ID NO: 11 - DNA, 42 bp, Artificial Sequence
  Synthetic nucleotide
ataagtgacgtactagcaacgaaggaaatgagaaaaatccag

SEQ ID NO: 12 - DNA, 20 bp, Artificial Sequence
  Synthetic nucleotide
ttgggactacaagagcactg

SEQ ID NO: 13 - DNA, 43 bp, Artificial Sequence
  Synthetic nucleotide
ataagtgacgtactagcaacgctccaaatgctagctatgttaa

SEQ ID NO: 14 - DNA, 23 bp, Artificial Sequence
  Synthetic nucleotide
gagagaagcaagtagtattttat

SEQ ID NO: 15 - DNA, 42 bp, Artificial Sequence
  Synthetic nucleotide
ataagtgacgtactagcaacgtgtcttgtgaaacaaaatgct

SEQ ID NO: 16 - DNA, 21 bp, Artificial Sequence
  Synthetic nucleotide
aaaccatgtcctctgtggaca

SEQ ID NO: 17 - DNA, 46 bp, Artificial Sequence
  Synthetic nucleotide
ataagtgacgtactagcaacggaacatttaaaaagttcagatgtta

SEQ ID NO: 18 - DNA, 20 bp, Artificial Sequence
  Synthetic nucleotide
tttaagacactctaacactt

SEQ ID NO: 19 - DNA, 41 bp, Artificial Sequence
  Synthetic nucleotide
ataagtgacgtactagcaacgactgactctctctgcctatt

SEQ ID NO: 20 - DNA, 28 bp, Artificial Sequence
  Synthetic nucleotide
cccataaatatgtataatgattatgtat

SEQ ID NO: 21 - DNA, 17 bp, Artificial Sequence
  Synthetic nucleotide
gtgacgtactagcaacg

SEQ ID NO: 22 - DNA, 23 bp, Artificial Sequence
  Synthetic nucleotide
ccctgatttggtcaatatgtgta